(19) United States
(12) Patent Application Publication
(10) Pub. No.: US 2009/0281385 A1
(43) Pub. Date: Nov. 12, 2009

(54) Endoscope Having Distal End Body Adapted to Plural Types of Light Distribution Lens Units

(75) Inventor: Takahiro HATOMA, Kanagawa (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/429,415

(22) Filed: Apr. 24, 2009

(30) Foreign Application Priority Data
May 8, 2008 (JP) 2008-121742
Feb. 3, 2009 (JP) 2009-022078

(51) Int. Cl. A61B 1/07; G02B 7/14; A61B 1/01

(52) U.S. Cl. 600/114; 359/819; 600/177

(57) ABSTRACT

An endoscope includes a distal end body provided at a distal end of an insertion part to be inserted into a body cavity, a light guide attached into the distal end body to emit light for illuminating the body cavity, a tube fitting hole provided in the distal end body such that any one of plural lens holding tubes is fitted therein, and a positioning step formed in the tube fitting hole to establish contact with any one of positioning end faces of the first lens holding tube and the second lens holding tube. The plural lens holding tubes hold respective lens units to diverge the light emitted, and have a common outer diameter and a common axial length defined as a distance between a distal end face and the positioning end face of each of the plural lens holding tubes.

### BACKGROUND OF THE INVENTION

The following description relates to one or more endoscopes having a distal end body adapted to a plurality of types of light distribution lens units.

In general, an endoscope does not have an enough distribution angle of illumination light to illuminate a region around the field of view, which illumination light is directly emitted by a light guide (more specifically, a light guide fiber bundle) through which the illumination light is transmitted. Therefore, a light distribution lens designed to broaden the light distribution angle is disposed to face a light emitting end face of the light guide that is located at the distal end of an insertion part of the endoscope.

As such a light distribution lens, a single concave lens is generally employed (for example, see Japanese Patent Provisional Publication No. 2005-152278). Nevertheless, in a type of endoscope required to diverge illumination light broadly and evenly over the region around the field of view, a light distribution lens group is utilized which includes a plurality of lenses (for example, see Japanese Patent Provisional Publication No. 2008-48980).

### SUMMARY OF THE INVENTION

In the case of an endoscope configured to observe an examined region having a wide cavity shape such as a stomach, a light distribution lens group including a convex lens is desired to be disposed to face the light emitting end face of the light guide such that illumination light is diverged broadly and evenly over the region around the field of view.

In the meantime, in the case of an endoscope configured to observe an examined region having an elongated luminal shape such as intestines, a short observation distance up to surrounding regions to be observed results in too bright illumination for the regions, whereas a long observation distance up to a central region to be observed results in dark illumination for the region. Hence, a single concave lens, which is designed to emit light with the light intensity of a central portion thereof higher than that of surrounding portions thereof, is desired to be placed to face the light emitting end face of the light guide.

Accordingly, for an endoscope for observing a stomach, a distal end body provided at the distal end of the insertion part of the endoscope has to be formed to get the light distribution lens group including the convex lens incorporated thereinto. Meanwhile, for an endoscope for observing intestines, the distal end body has to be formed to get the single convex lens incorporated thereinto. However, it is economically inefficient to manufacture two types of distal end bodies that differ only as to their portions into which respective different light distribution lens units are incorporated.

Aspects of the present invention are advantageous to provide one or more improved endoscopes having a distal end body adapted such that any one of plural types of light distribution lens units is incorporated thereinto.

According to aspects of the present invention, an endoscope is provided which includes a distal end body provided at a distal end of an insertion part adapted to be inserted into a body cavity, a light guide attached into the distal end body, the light guide being configured to emit light for illuminating the body cavity from a light emitting end face thereof, a tube fitting hole provided in the distal end body, the tube fitting hole being configured such that any one of a first lens holding tube and a second lens holding tube is fitted therein, and a positioning step formed in the tube fitting hole, the positioning step being configured to establish contact with any one of a first positioning end face of the first lens holding tube and a second positioning end face of the second lens holding tube. The first lens holding tube holds a first lens unit to diverge the light emitted from the light emitting end face of the light guide, and has a first outer diameter and a first axial length defined as a distance between a distal end face of the first lens unit and the first positioning end face of the first lens holding tube. The second lens holding tube holds a second lens unit to diverge the light emitted from the light emitting end face of the light guide, and has a second outer diameter and a second axial length defined as a distance between a distal end face of the second lens unit and the second positioning end face of the second lens holding tube. The first outer diameter is substantially equal to the second outer diameter, and the first axial length is substantially equal to the second axial length.

Optionally, the first positioning end face of the first lens holding tube may be configured to, when the first lens holding tube is fitted in the tube fitting hole, determine a position of the first lens holding tube relative to the distal end body in a first axis line direction of the first lens holding tube. In this case, the second positioning end face of the second lens holding tube may be configured to, when the second lens holding tube is fitted in the tube fitting hole, determine a position of the second lens holding tube relative to the distal end body in a second axis line direction of the second lens holding tube.

Optionally, the endoscope may further include an escape hole formed following the tube fitting hole, the escape hole being configured to, when the second lens holding tube is fitted in the tube fitting hole, loosely accommodate a portion, of the second lens unit or the second lens holding tube, which protrudes rearward from the second positioning end face of the second lens holding tube.

Optionally, the first lens holding tube may be fitted in the tube fitting hole.

Further optionally, the rear end face of the first lens unit is located in the first lens holding tube, and the light guide may be attached to the distal end body such that the light emitting end face thereof is inserted in the first lens holding tube.

Still optionally, a rear end face of the first lens holding tube may be configured as the first positioning end face to determine the position of the first lens holding tube relative to the distal end body in the first axis line direction in contact with the positioning step.

Yet optionally, the first lens unit may include a single concave lens.

Alternatively, the second lens holding tube may be fitted in the tube fitting hole.

Still optionally, a rear end face of the second lens unit may be located in a position corresponding to a rear end face of the second lens holding tube in a second axis line direction of the second lens holding tube. In this case, the light guide may be attached to the distal end body such that the light emitting end face thereof is placed in the position corresponding to the rear end face of the second lens holding tube in the second axis line direction.

Further optionally, the second positioning end face of the second lens holding tube may be formed in a middle of an outer circumferential portion of the second lens holding tube. Furthermore, the endoscope may further include an escape hole formed behind the positioning step in the second axis line direction, the escape hole being configured to loosely accommodate a portion of the second lens holding tube that protrudes rearward from the second positioning end face of the second lens holding tube.

Yet optionally, a rear end face of the second lens unit may be located behind a rear end face of the second lens holding tube in a second axis line direction of the second lens holding tube, and the light guide may be attached to the distal end body such that the light emitting end face thereof is placed outside the second lens holding tube.

Optionally, the rear end face of the second lens holding tube may be configured as the second positioning end face to determine the position of the second lens holding tube relative to the distal end body in the second axis line direction in contact with the positioning step.

Further optionally, the endoscope may further include an escape hole formed behind the positioning step in the second axis line direction, the escape hole being configured to loosely accommodate a portion of the second lens unit that protrudes rearward from the second positioning end face of the second lens holding tube.

Furthermore, the second lens unit may be configured as a lens group including a convex lens and at least one additional lens.

According to aspects of the present invention, further provided is an endoscope, which includes a distal end body provided at a distal end of an insertion part adapted to be inserted into a body cavity, a light guide attached into the distal end body, the light guide being configured to emit light for illuminating the body cavity, a tube fitting hole provided in the distal end body, the tube fitting hole being configured such that any one of plural lens holding tubes is fitted therein, and a positioning step formed in the tube fitting hole, the positioning step being configured to establish contact with any one of a positioning end face of the first lens holding tube and a positioning end face of the second lens holding tube. The plural lens holding tubes hold respective lens units to diverge the light emitted by the light guide, and have a common outer diameter and a common axial length defined as a distance between a distal end face of each of the lens units and the positioning end face of each of the plural lens holding tubes.

According to aspects of the present invention, further provided is an endoscope, which includes a distal end body provided at a distal end of an insertion part adapted to be inserted into a body cavity, a light guide attached into the distal end body, the light guide being configured to emit light for illuminating the body cavity, a tube fitting hole provided in the distal end body, the tube fitting hole being configured such that any one of a first lens holding tube and a second lens holding tube is fitted therein, and a positioning step formed in the tube fitting hole, the positioning step being configured to establish contact with any one of a first positioning end face of the first lens holding tube and a second positioning end face of the second lens holding tube. The first lens holding tube holds a first lens unit to diverge the light emitted by the light guide, and has a first outer diameter and a first axial length defined as a distance between a distal end face of the first lens unit and the first positioning end face of the first lens holding tube. The second lens holding tube holds a second lens unit to diverge the light emitted by the light guide, and has a second outer diameter and a second axial length defined as a distance between a distal end face of the second lens unit and the second end face of the second lens holding tube. The first outer diameter is substantially equal to the second outer diameter, and the first axial length is substantially equal to the second axial length. The first positioning end face of the first lens holding tube is configured to, when the first lens holding tube is fitted in the tube fitting hole, determine a position of the first lens holding tube relative to the distal end body in a first axis line direction of the first lens holding tube. The second positioning end face of the second lens holding tube is configured to, when the second lens holding tube is fitted in the tube fitting hole, determine a position of the second lens holding tube relative to the distal end body in a second axis line direction of the second lens holding tube. The endoscope further includes an escape hole formed following the tube fitting hole, the escape hole being configured to, when the second lens holding tube is fitted in the tube fitting hole, loosely accommodate a portion, of the second lens unit or the second lens holding tube, which protrudes rearward from the second positioning end face of the second lens holding tube.

### BRIEF DESCRIPTION OF THE ACCOMPANYING DRAWINGS

FIG. 1 is a cross-sectional side view of a distal end portion of an insertion part of an endoscope for observing intestines in a first embodiment according to one or more aspects of the present invention.

FIG. 2 is a cross-sectional side view of a distal end portion of an insertion part of an endoscope for observing a stomach in the first embodiment according to one or more aspects of the present invention.

FIG. 3 is a cross-sectional side view showing a single light distribution lens attached to a single light distribution lens holding tube in the first embodiment according to one or more aspects of the present invention.

FIG. 4 is a cross-sectional side view showing a light distribution lens group attached to a light distribution lens group holding tube in the first embodiment according to one or more aspects of the present invention.

FIG. 5 is a cross-sectional side view of a distal end body in the first embodiment according to one or more aspects of the present invention.

FIG. 6 is a cross-sectional side view of a distal end portion of an insertion part of an endoscope for observing a stomach in a second embodiment according to one or more aspects of the present invention.

FIG. 7 is a cross-sectional side view showing the light distribution lens group attached to a light distribution lens group holding tube in the second embodiment according to one or more aspects of the present invention.

### DETAILED DESCRIPTION OF THE EMBODIMENTS

It is noted that various connections are set forth between elements in the following description. It is noted that these connections in general and, unless specified otherwise, may be direct or indirect and that this specification is not intended to be limiting in this respect.

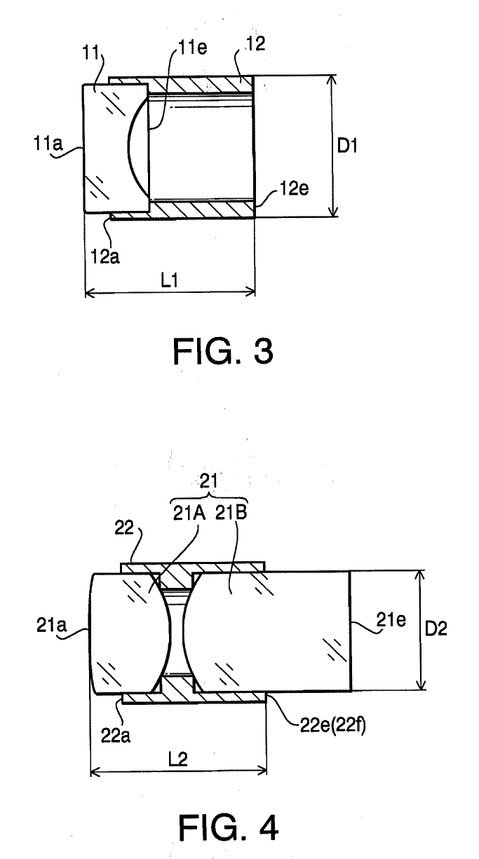

Hereinafter, embodiments according to aspects of the present invention will be set forth with reference to the accompanying drawings. FIG. 3 shows a single light distribution lens 11 held by a single light distribution lens holding tube 12 which is made of metal and formed substantially in a cylindrical shape in a first embodiment according to aspects of the present invention.

The single light distribution lens 11 includes a single concave lens, which has a distal end face 11a formed to be flat at a side exposed outside and a rear end face 11e formed in a concave shape. When being placed to face a light emitting face of a light guide through which illumination light is transmitted, the single light distribution lens 11 gives a widened light distribution angle for light transmitted therethrough with the light intensity of a central portion of the light much higher than that of surrounding portions thereof.

It is noted that the distal end face 11a of the single light distribution lens 11 may not necessarily be flat. Additionally, the single light distribution lens 11 has only to be substantially a single concave lens. For example, the single light distribution lens 11 may include a so-called cemented lens with a plurality of lenses put together into a single lens.

The rear end face 11e of the single light distribution lens 11 is located in the single light distribution lens holding tube 12. Further, the distal end face 11a of the single light distribution lens 11 is configured to protrude forward from a distal end face 12a of the single light distribution lens holding tube 12.

FIG. 4 shows a light distribution lens group 21 held by a light distribution lens group holding tube 22 which is made of metal and formed substantially in a cylindrical shape. The light distribution lens group 21 consists of a plurality of lenses 21A and 21B including a convex lens 21A. Specifically, in the first embodiment, the light distribution lens group 21 includes the convex lens 21A disposed at the distal end thereof and a glass rod 21B disposed at the rear end thereof with a core surrounded by a clad.

A distal end face of the glass rod 21B is formed in a convex shape, and a rear end face 21e of the glass rod 21B is formed to be flat. When being placed to face the light emitting face of the light guide, the light distribution lens group 21 gives a broadened light distribution angle for light transmitted therethrough with the light intensity of a central portion of the light less different from that of surrounding portions thereof.

It is noted that the glass rod 21B may be configured with a structure other than the aforementioned structure, and that the light distribution lens group 21 may be configured with three or more lenses. The rear end face 21e of the glass rod 21B is configured to protrude rearward from a rear end face 22e of the light distribution lens group holding tube 22. In addition, a distal end face 21a of the convex lens 21A is configured to protrude forward from a distal end face 22a of the light distribution lens group holding tube 22. It is noted that as described below, here, the rear end face 22e of the light distribution lens group holding tube 22 serves as a positioning end face 22f.

The single light distribution lens holding tube 12 and the light distribution lens group holding tube 22 have substantially the same outer diameter (i.e., D1=D2 or D1≈D2). Furthermore, a distance L1 between the distal end face 11a of the single light distribution lens 11 and the rear end face 12e of the single light distribution lens holding tube 12 is configured to be substantially equal to a distance L2 between the distal end face 21a of the light distribution lens group 21 and the rear end face 22e of the light distribution lens group holding tube 22 (i.e., L1=L2 or L1≈L2).

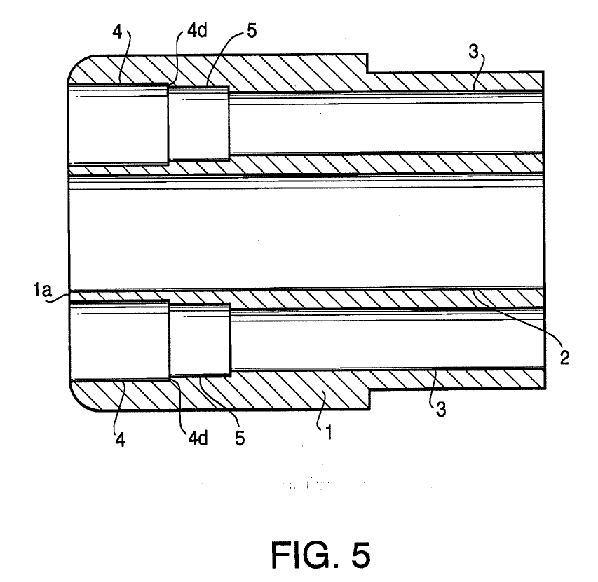

FIG. 5 schematically shows a distal end body 1 made of electrical insulating plastic material, which body is included in an insertion part of an endoscope. The distal end body 1 includes an imaging unit hole 2, in which an observation optical system is located, formed in a penetrating fashion in a direction parallel to an axis line of the distal end body 1.

Further, the distal end body 1 has two light guide attachment holes 3 formed in parallel with the imaging unit hole 2. The light guide attachment holes 3 is configured such that a light emitting end face of a below-mentioned light guide fiber bundle is inserted thereinto from the rear end of the distal end body 1 and fixed.

Furthermore, the distal end body 1 includes holding tube fitting holes 4 each of which is provided at the side of the distal end of each light guide attachment hole 3 on the same axis line as that of each light guide attachment hole 3, so as to form a train of holes with each light guide attachment hole 3. The holding tube fitting holes 4 are configured so that any of the single light distribution lens holding tube 12 and the light distribution lens group holding tube 22 can be fitted thereinto from the side of a distal end face 1a of the distal end body 1.

Additionally, the distal end body 1 has a positioning step 4d formed at the rear end of each of the holding tube fitting holes 4. The positioning step 4d is configured to establish contact with the rear end face 12e or 22e of the holding tube 12 or 22 fitted into each of the holding tube fitting holes 4.

Moreover, an escape hole 5 is formed following each of the holding tube fitting holes 4. The escape hole 5 is configured to loosely accommodate a portion of the light distribution lens group 21 that protrudes rearward from the rear end face 22e of the light distribution lens group tube 22. It is noted that the distal end body 1 further includes other portions formed therein (not shown) such as a treatment tool channel, an air supply channel, and a water supply channel.

The distal end body 1 configured as above can get any of the single light distribution lens holding tube 12 and the light distribution lens group tube 22 fitted into the holding tube fitting holes 4 thereof. Therefore, the distal end body 1 can commonly be used for both an endoscope suitable for observing a stomach with the single light distribution lens 11 and an endoscope suitable for observing intestines with the light distribution lens group 21.

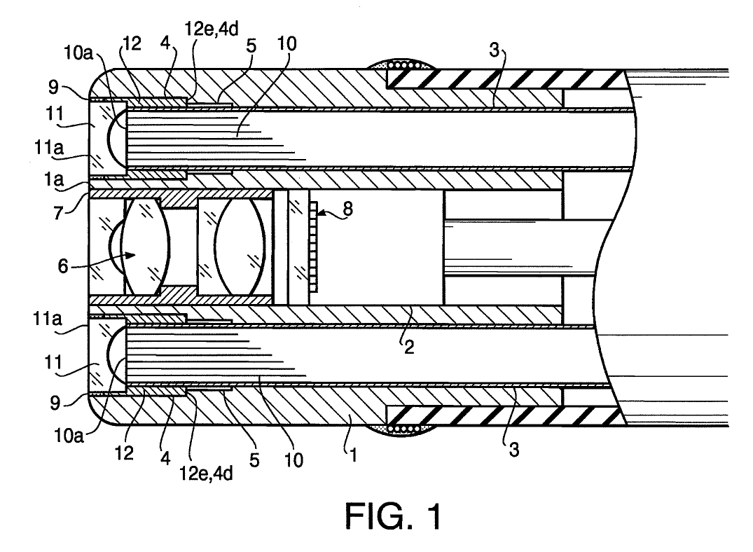

FIG. 1 illustrates a distal end portion of an insertion part of an endoscope for observing intestines (a first type of endoscope) that uses the single light distribution lens 11. There are disposed and fixed with bonding material in the imaging unit hole 2 of the distal end body 1, an objective lens barrel 7 that supports an objective optical system 6 as a unit, and a solid-state image sensor 8.

The single light distribution lens holding tube 12 is bonded and fixed within each of the holding tube fitting holes 4. In this situation, the contact between the rear end face 12e of the single light distribution lens holding tube 12 and the positioning step 4d of the holding tube fitting hole 4 determines the position of the single light distribution lens holding tube 12 relative to the distal end body 1.

At this time, the distal end face 11a of the single light distribution lens 11 is substantially in the same level as the distal end face 1a of the distal end body 1, or slightly protrudes forward from the distal end face 1a of the distal end body 1. Each recess, formed between the distal end face 1a of the distal end body 1 and the distal end face 12a of the single light distribution lens holding tube 12, is filled with electrical insulating bond 9.

A light guide fiber bundle 10 is laid through the light guide attachment hole 3 of the distal end body 1 and fixed there. In this situation, a light emitting end face 10a of the light guide fiber bundle 10 is inserted into the single light distribution lens holding tube 12 and placed to closely face the rear end face 11e of the single light distribution lens 11. Consequently, it is possible to obtain such light distribution characteristics that illumination light, emitted by the light emitting end face 10a of the light guide fiber bundle 10, is broadened through the single light distribution lens 11 with the light intensity of a central portion of the light higher than that of surrounding portions thereof.

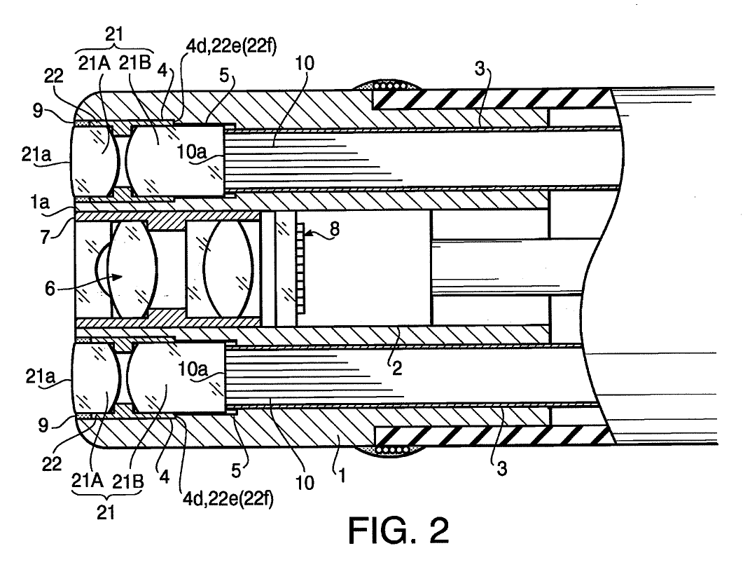

FIG. 2 illustrates the distal end of an insertion part of an endoscope for observing a stomach (a second type of endoscope) that uses the light distribution lens group 21. In the same manner as the first type of endoscope for observing intestines, the objective lens barrel 7 that supports the objective optical system 6 as a unit and the solid-state image sensor 8 are disposed and fixed with bonding material in the imaging unit hole 2 of the distal end body 1.

The light distribution lens group holding tube 22 is bonded and fixed within each of the holding tube fitting holes 4. In this situation, the contact between the rear end face 22e of the light distribution lens group holding tube 22 and the positioning step 4d of the holding tube fitting hole 4 determines the position of the light distribution lens group holding tube 22 relative to the distal end body 1. Accordingly, the rear end face 22e of the light distribution lens group holding tube 22 serves as the positioning end face 22f to determine the position of the light distribution lens group holding tube 22 relative to the distal end body 1.

At this time, the distal end face 21a of the light distribution lens group 21 is substantially in the same level as the distal end face 1 a of the distal end body 1, or slightly protrudes forward from the distal end face 1a of the distal end body 1. Each recess, formed between the distal end face 1a of the distal end body 1 and the distal end face 22a of the light distribution lens group holding tube 22, is filled with the electrical insulating bond 9.

The light guide fiber bundle 10 is laid through the light guide attachment hole 3 of the distal end body 1 and fixed there. In this situation, the light emitting end face 10a of the light guide fiber bundle 10 is attached in contact with the rear end face 21e of the light distribution lens group 21 in the state not to be inserted into the light distribution lens group holding tube 22. Consequently, it is possible to obtain such light distribution characteristics that illumination light, emitted by the light emitting end face 10a of the light guide fiber bundle 10, is broadened through the single light distribution lens 11 with the light intensity of a central portion of the light less different from that of surrounding portions thereof.

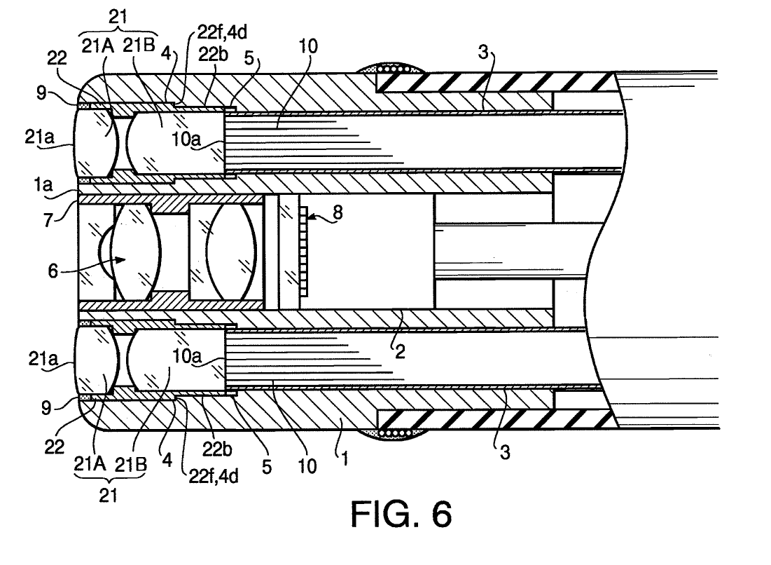

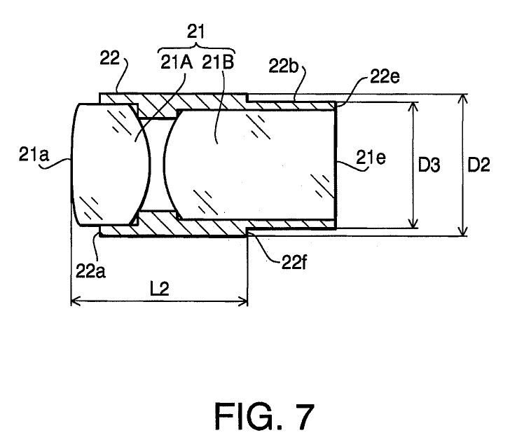

FIG. 6 is a cross-sectional side view of a distal end portion of an insertion part for the second type of endoscope for observing a stomach in a second embodiment according to aspects of the present invention. FIG. 7 is a cross-sectional side view showing the light distribution lens group 21 attached to a light distribution lens group holding tube 22 in the second embodiment.

In the second embodiment, only the shape of the light distribution lens group holding tube 22 is different from that in the first embodiment. It is noted that the other configurations, including the configurations of the distal end body 1 and the single light distribution lens holding tube 12, are the same as those in the first embodiment, and thus illustrations and explanations therefor will be omitted.

In the second embodiment, the light distribution lens group 21 is the same as that in the first embodiment. The light distribution lens group holding tube 22 is configured to be longer in the axis line direction thereof than that in the first embodiment, such that the rear end face 21e of the light distribution lens group 21 is in the same position as the rear end face 22e of the light distribution lens group holding tube 22 in the axis line direction of the light distribution lens group holding tube 22. Consequently, there are less risks that the light distribution lens group 21 to be attached into the light distribution lens group holding tube 22 is damaged in a conveying process or an assembly process for the light distribution lens group 21, and thus a fabrication yield of the endoscope is boosted.

The positioning end face 22f, for determining the position of the light distribution lens group holding tube 22 relative to the distal end body 1, is formed in the middle of an outer circumferential portion of the light distribution lens group holding tube 22. Further, the positioning end face 22f is configured to establish contact with the positioning step 4d formed in the holding tube fitting hole 4.

The aforementioned escape hole 5 is configured to loosely accommodate a portion 22b of the light distribution lens group holding tube 22 that protrudes rearward from the positioning end face 22f. An outer diameter D3 of the protruding portion 22b is set such that an outer circumferential surface of the protruding portion 22b does not contact an inner circumferential surface of the escape hole 5.

Meanwhile, when the light distribution lens group holding tube 22 is used for the second type of endoscope, the light distribution lens group holding tube 22 is attached into the distal end body 1 such that the light emitting end face 10a of the light guide fiber bundle 10 establishes contact with the rear end face 21e of the light distribution lens group 21 in a position corresponding to the rear end face 22e of the light distribution lens group holding tube 22 in the axis line direction of the light distribution lens group holding tube 22.

Hereinabove, the embodiments according to aspects of the present invention have been described. The present invention can be practiced by employing conventional materials, methodology and equipment. Accordingly, the details of such materials, equipment and methodology are not set forth herein in detail. In the previous descriptions, numerous specific details are set forth, such as specific materials, structures, processes, etc., in order to provide a thorough understanding of the present invention. However, it should be recognized that the present invention can be practiced without reapportioning to the details specifically set forth. In other instances, well known processing structures have not been described in detail, in order not to unnecessarily obscure the present invention.

Only exemplary embodiments of the present invention and but a few examples of its versatility are shown and described in the present disclosure. It is to be understood that the present invention is capable of use in various other combinations and environments and is capable of changes or modifications within the scope of the inventive concept as expressed herein.

The present disclosure relates to the subject matter contained in Japanese Patent Applications No. P2008-121742 filed on May 8, 2008 and No. P2009-022078 filed on Feb. 3, 2009, which is expressly incorporated herein by reference in its entirety.

What is claimed is:

1. An endoscope comprising:
a distal end body provided at a distal end of an insertion part adapted to be inserted into a body cavity;
a light guide attached into the distal end body, the light guide being configured to emit light for illuminating the body cavity from a light emitting end face thereof;
a tube fitting hole provided in the distal end body, the tube fitting hole being configured such that any one of a first lens holding tube and a second lens holding tube is fitted therein, and
a positioning step formed in the tube fitting hole, the positioning step being configured to establish contact with any one of a first positioning end face of the first lens holding tube and a second positioning end face of the second lens holding tube,
wherein the first lens holding tube holds a first lens unit to diverge the light emitted from the light emitting end face of the light guide, and has a first outer diameter and a first axial length defined as a distance between a distal end face of the first lens unit and the first positioning end face of the first lens holding tube,
wherein the second lens holding tube holds a second lens unit to diverge the light emitted from the light emitting end face of the light guide, and has a second outer diameter and a second axial length defined as a distance between a distal end face of the second lens unit and the second positioning end face of the second lens holding tube, and
wherein the first outer diameter is substantially equal to the second outer diameter, and the first axial length is substantially equal to the second axial length.

2. The endoscope according to claim 1,
wherein the first positioning end face of the first lens holding tube is configured to, when the first lens holding tube is fitted in the tube fitting hole, determine a position of the first lens holding tube relative to the distal end body in a first axis line direction of the first lens holding tube, and
wherein the second positioning end face of the second lens holding tube is configured to, when the second lens holding tube is fitted in the tube fitting hole, determine a position of the second lens holding tube relative to the distal end body in a second axis line direction of the second lens holding tube.

3. The endoscope according to claim 1, further comprising an escape hole formed following the tube fitting hole, the escape hole being configured to, when the second lens holding tube is fitted in the tube fitting hole, loosely accommodate a portion, of the second lens unit or the second lens holding tube, which protrudes rearward from the second positioning end face of the second lens holding tube.

4. The endoscope according to claim 1,
wherein the first lens holding tube is fitted in the tube fitting hole.

5. The endoscope according to claim 4,
wherein a rear end face of the first lens unit is located in the first lens holding tube, and
wherein the light guide is attached to the distal end body such that the light emitting end face thereof is inserted in the first lens holding tube.

6. The endoscope according to claim 4,
wherein a rear end face of the first lens holding tube is configured as the first positioning end face to determine the position of the first lens holding tube relative to the distal end body in the first axis line direction in contact with the positioning step.

7. The endoscope according to claim 4,
wherein the first lens unit includes a single concave lens.

8. The endoscope according to claim 1,
wherein the second lens holding tube is fitted in the tube fitting hole.

9. The endoscope according to claim 8,
wherein a rear end face of the second lens unit is located in a position corresponding to a rear end face of the second lens holding tube in a second axis line direction of the second lens holding tube, and
wherein the light guide is attached to the distal end body such that the light emitting end face thereof is placed in the position corresponding to the rear end face of the second lens holding tube in the second axis line direction.

10. The endoscope according to claim 9,
wherein the second positioning end face of the second lens holding tube is formed in a middle of an outer circumferential portion of the second lens holding tube, and
wherein the endoscope further comprises an escape hole formed behind the positioning step in the second axis line direction, the escape hole being configured to loosely accommodate a portion of the second lens holding tube that protrudes rearward from the second positioning end face of the second lens holding tube.

11. The endoscope according to claim 8,
wherein a rear end face of the second lens unit is located behind a rear end face of the second lens holding tube in a second axis line direction of the second lens holding tube, and
wherein the light guide is attached to the distal end body such that the light emitting end face thereof is placed outside the second lens holding tube.

12. The endoscope according to claim 11,
wherein the rear end face of the second lens holding tube is configured as the second positioning end face to determine the position of the second lens holding tube relative to the distal end body in the second axis line direction in contact with the positioning step.

13. The endoscope according to claim 12,
wherein the endoscope further comprises an escape hole formed behind the positioning step in the second axis line direction, the escape hole being configured to loosely accommodate a portion of the second lens unit that protrudes rearward from the second positioning end face of the second lens holding tube.

14. The endoscope according to claim 8,
wherein the second lens unit is configured as a lens group including a convex lens and at least one additional lens.

15. An endoscope comprising:
a distal end body provided at a distal end of an insertion part adapted to be inserted into a body cavity;
a light guide attached into the distal end body, the light guide being configured to emit light for illuminating the body cavity;
a tube fitting hole provided in the distal end body, the tube fitting hole being configured such that any one of plural lens holding tubes is fitted therein; and
a positioning step formed in the tube fitting hole, the positioning step being configured to establish contact with any one of a positioning end face of the first lens holding tube and a positioning end face of the second lens holding tube,
wherein the plural lens holding tubes hold respective lens units to diverge the light emitted by the light guide, and have a common outer diameter and a common axial length defined as a distance between a distal end face of each of the lens units and the positioning end face of each of the plural lens holding tubes.

16. An endoscope comprising:
a distal end body provided at a distal end of an insertion part adapted to be inserted into a body cavity;
a light guide attached into the distal end body, the light guide being configured to emit light for illuminating the body cavity;
a tube fitting hole provided in the distal end body, the tube fitting hole being configured such that any one of a first lens holding tube and a second lens holding tube is fitted therein; and
a positioning step formed in the tube fitting hole, the positioning step being configured to establish contact with any one of a first positioning end face of the first lens holding tube and a second positioning end face of the second lens holding tube,
wherein the first lens holding tube holds a first lens unit to diverge the light emitted by the light guide, and has a first outer diameter and a first axial length defined as a distance between a distal end face of the first lens unit and the first positioning end face of the first lens holding tube,
wherein the second lens holding tube holds a second lens unit to diverge the light emitted by the light guide, and has a second outer diameter and a second axial length defined as a distance between a distal end face of the second lens unit and the second end face of the second lens holding tube,
wherein the first outer diameter is substantially equal to the second outer diameter, and the first axial length is substantially equal to the second axial length,
wherein the first positioning end face of the first lens holding tube is configured to, when the first lens holding tube is fitted in the tube fitting hole, determine a position of the first lens holding tube relative to the distal end body in a first axis line direction of the first lens holding tube,
wherein the second positioning end face of the second lens holding tube is configured to, when the second lens holding tube is fitted in the tube fitting hole, determine a position of the second lens holding tube relative to the distal end body in a second axis line direction of the second lens holding tube, and
wherein the endoscope further comprises an escape hole formed following the tube fitting hole, the escape hole being configured to, when the second lens holding tube is fitted in the tube fitting hole, loosely accommodate a portion, of the second lens unit or the second lens holding tube, which protrudes rearward from the second positioning end face of the second lens holding tube.
